(19) United States
(12) Patent Application Publication
(10) Pub. No.: US 2008/0114080 A1
(43) Pub. Date: May 15, 2008

(54) Compositions and Methods for the Treatment of Cystic Fibrosis and other Pulmonary Disorders

(76) Inventor: Hao Chen, Columbia, MD (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/757,292

(22) Filed: Jun. 1, 2007

Related U.S. Application Data
(60) Provisional application No. 60/803,838, filed on Jun. 2, 2006.

(51) Int. Cl. C12Q 1/68; A61K 47/00

(52) U.S. Cl. 514/789; 435/6

(57) ABSTRACT

A screening assay to identify agents which act synergistically to compensate for defective chloride ion transport in cells harboring a mutation in the CFTR in provided.

This application claims priority to U.S. provisional application, 60/803,838 filed Jun. 2, 2006, the entire contents of which is incorporated herein by reference.

Pursuant to 35 U.S.C. §202(c) it is acknowledged that the U.S. Government has certain rights in the invention described, which was made in part with funds from the National Institutes of Health, Grant Number, IR43HL078070-01A1

### FIELD OF THE INVENTION

The present invention relates to the fields of pulmonary disease and signal transduction. More specifically, the invention provides methods and compositions useful for the treatment of cystic fibrosis and other forms of pulmonary dysfunction.

### BACKGROUND OF THE INVENTION

Several publications and patent documents are cited throughout the specification in order to describe the state of the art to which this invention pertains. Each of these citations is incorporated herein by reference as though set forth in full.

Cystic fibrosis (CF) is one of the most lethal of the human monogenetic diseases and is responsible for approximately 30,000 deaths of children and young adults in the United States each year. The heterozygous genetic carrier frequency in the Caucasian population is 1 person in 25 and that number of carriers results in an average of 1 case of CF disease in every 2,500 newborns. According to the CF Foundations' National Patient Registry, the median age of survival for a person with cystic fibrosis is about 33 years, a most disheartening figure for the patients, for the parents and for society as a whole (Kulich, 2003).

Several gene therapy trials for the treatment of cystic fibrosis have been conducted (Griesenbach et al., 2002; Brown, 2002; Griesenbach, 2003). To date, these trial results have been largely inconclusive as to whether these therapies were beneficial and effective in treating the disease. At present, the efficacy of the gene therapy approach is generally low (Check, 2003). New medications capable of alleviating the symptoms of CF are still urgently needed.

Medications such as tobramycin and azithromycin are employed to treat the co-morbid bacterial infections often associated with CF; whereas the drug, pulmozyme, acts by thinning the mucus secretions in the lungs to improve the quality of life of the patients. A recent report indicates that CPX, an adenosine A1 antagonist, is somewhat effective in treating mild cystic fibrosis (McCarty et al, 2002). cDNA microarray analysis indicates that exposure to CPX can induce substantial changes in gene expression in cells transfected with mutant and wild type CFTR. Perhaps as a beneficial indication, some changes in the gene expression profile move the “mutant form expression profile” closer to the profile of the wild type (Srivastava et al, 1999).

The gene responsible for the disease phenotype of cystic fibrosis (impaired ion transport across epithelia) encodes a protein called cystic fibrosis transmembrane conductance regulator (CFTR). The wild type CFTR, located primarily in the apical membrane of the epithelium, plays a crucial role in epithelium salt transport, fluid management and regulation of ion concentration and electrolyte secretion. The ion transport function of CFTR in epithelial cells is ATP-gated and cAMP-dependent.

Mutations of the CFTR protein, typically a deletion of a phenylalanine at position 508 (ΔF508), disable the ion transport activity. A majority of the mutated protein never reaches the apical membrane (Denning, 1992). The lack of functional ion transport induces a constellation of phenotypic abnormalities.

Chloride channels are well known and the phenotypic properties of Cl⁻ channels have been extensively studied. In a recent report, Shimizu et al. (Shimizu, 2000) pointed out the presence of volume-sensitive Cl⁻ channels in human epithelial cells. These ion channel activities may be mediated through changes in intracellular Ca⁺⁺ concentration, which are often coupled to stimulation of membrane receptors.

Unlike the pharmaceutical successes in discovering drugs active at membrane receptors and cation channels, there has not been a substantial body of small molecules found to be capable of opening CFTR (Schultz et al, 1999). In fact, since the majority of mutated CFTR protein is being sequestered and is absent from the apical membrane, it is quite possible that most of the compounds that will be effective in CF models may act by forcing chloride transport via indirect cellular mechanisms. There are a limited number of compounds (8 different structural classes) known to be CFTR channel openers. Little is known with certainty whether these effects are the result of direct molecular interactions between the small molecules and the mutated ion channels or if they are indirect effectors.

Clearly, a need exists for improved treatments for this devastating disease. It is an object of this invention to provide such treatments.

### SUMMARY OF THE INVENTION

In accordance with the present invention, a method for identifying agents which compensate for the defective ion transport of the mutated CFTR in a cell is provided. An exemplary method entails providing a plurality of cell lines, at least one cell line comprising a mutation in the CFTR gene and least one cell line being wild type for CFTR expression; contacting the cell lines with at least one agent which modulates intracellular messenger activity, and assessing the cell lines of for an alteration in chloride secretion following incubation in the presence of said agent, agents which increase said chloride ion transport in cells comprising a mutation in the CFTR being effective for the treatment of cystic fibrosis. The agent may modulate cellular signaling molecules, which include, without limitation, G-protein coupled receptors, cAMP, Ca⁺⁺, ion channels or any of the molecules listed in Table 2.

In a preferred embodiment of the invention, the cell lines are contacted with two agents simultaneously and said cells are assessed to determine if said agents act synergistically to increase chloride ion transport. The invention also includes the combination of agents so identified. Identification of the effective combination of agents facilitates the preparation of a pharmaceutical composition comprising the combination of agents in a biologically acceptable carrier. Such pharmaceutical compositions are also within the scope of the present invention.

### BRIEF DESCRIPTION OF THE DRAWINGS

FIG. 1A shows a diagram depicting the five domains of the CFTR membrane protein (Sheppard 1999). FIG. 1B is a schematic diagram of a G protein-coupled receptor.

FIG. 2 depicts a schematic diagram of the multiple signal transduction pathways implicated in wild type CFTR regulation.

FIG. 3 shows that cellular Cl⁻ secretion is made possible by different transport mechanisms. Some may be through CFTR (PKA-PKC dependent); others may be regulated via Ca²⁺; or coupled to other cation transporters.

FIGS. 4A and 4B are histograms of FACS analysis which indicate the presence of CXCR2/CXCR2B (both isoforms of IL-8 receptors IL8Ra and b) in Calu-3, a lung adenocarcinoma line.

FIG. 5 is a graph showing the Calu-3 GPCR response profile to a collection of 85 small molecule receptor agonists.

FIG. 6 is a graph showing that C5a inhibits adenylyl cyclase activity in Calu-3 cells.

FIG. 7 is a graph showing that C5a inhibits basal Cl⁻ efflux via C5aR (CD88).

FIG. 8 is a graph showing that C5a inhibits cAMP dependent Cl— efflux.

FIG. 9 is a graph showing that IL-8 inhibits basal levels of Cl— transport via chemokine receptors (CXCR2/b) in Calu-3 cells.

FIG. 10 is a graph showing that IL-8 inhibits cAMP dependent Cl— efflux.

FIGS. 11A and 11B are graphs demonstrating that certain agonists and Gsα stimulation will reduce IL-8 secretion. However,certain receptors are more effective than others.

FIG. 12 is a graph demonstrating that targeting different receptors can result in synergistic enhancement of Cl— efflux.

### DETAILED DESCRIPTION OF THE INVENTION

The limited understanding of CFTR pharmacology may be attributed to several reasons. (1) The limited extent of CFTR mutation characterization beyond the known and characterized ΔF508 (ΔF508 is the most common form of mutations, representing about 70% of CF cases). There are more than 1,000 additional mutant alleles suspected to be associated with CF. (2) The heterogeneous CF system confounds the process of drug development. Generally, a variety of cells, recombinant and native cells of different origins (and CFTR expression levels), are used in different studies. The results or the interpretation of experimental observations may differ due to the differences in the combined effects of mutations and cell origins (differences in cells used for the cloning, expression levels or the tissue origin of the primary cell lines). (3) The limited knowledge of the cellular system (environment) in which the CFTR operates. Only very few studies comprehensively characterize the biochemical environment of the CFTR, such as the presence of other receptors (GPCRs and their coupled G-proteins), ion channels, and enzymes that may participate in regulating CFTR function, or Cl⁻ secretion via other mechanisms.

In contrast to our limited knowledge about the pharmacology directly effecting CFTR (mutated allele and normal wild type), there has been a great deal of study on CFTR structure and function (as a normal chloride channel, for review see Sheppard and Welsh, 1999), its mechanism of anion conduction (for review see Dawson et al, 1999) and its regulated gating by phosphorylation and nucleotide hydrolysis (for review see Gadsby and Nairn, 1999).

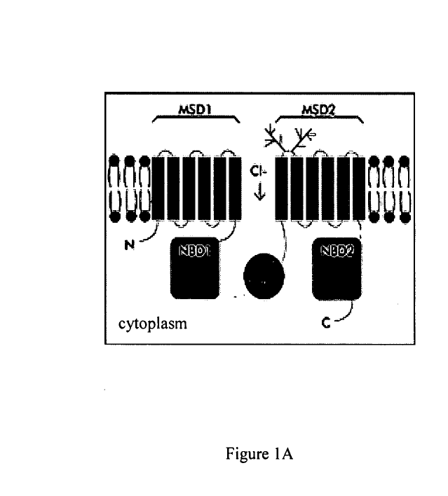

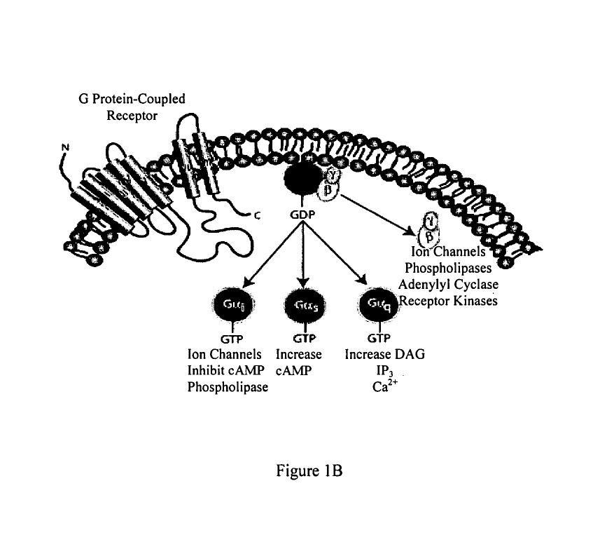

In accordance with the present invention, a novel approach for compensating for the defective chloride transport function of the cystic fibrosis transmembrane conductance regulator (CFTR; FIG. 1A) is provided. Mutations in the CFTR protein, typically the deletion of a phenylalanine at position 508 (the ΔF508 allele), disable the ion transport activity of CFTR (Denning, 1992; Gelman and Kopito, 2002). The lack of a functional ion transport mechanism induces a constellation of disease symptoms that are characteristic of CF. Compostions and methods which are effective to modulate the activities of membrane proteins other than CFTR are disclosed herein which should induce sufficient ion transport function thereby alleviating these disease symptoms. The compositions and methods described herein are able to recapitulate a near wild-type phenotype in CF-related cells by employing unrelated receptor/ligand interactions which overcome the CFTR abnormalities, thereby providing efficacious medications for this disease.

Most cellular ion channel activities are modulated via intracellular messengers, like cAMP and Ca++. The levels of these messengers can be modulated via GPCRs. There is an extensive body of pharmacological and biochemical knowledge available for these membrane proteins; whereas little is known about ligands directly regulating ion transport activity of CFTR. Identification of compounds which are capable of stimulating chloride secretion indirectly by modulating the activity of such receptors activities may provide immediate relief for the cystic fibrosis patient.

Recent advances have made it increasingly apparent that receptor signal transduction is a complicated and interactive process. The activity of co-expressed receptors and their respective signal transduction pathways may be regulated via more complex patterns within cells. Individual receptor activation and inactivation are interdependent on the activities of other coexisting (Werry et al, 2003) cellular components. Based on the receptor profiles provided herein, we anticipate that we will be able to influence cellular ion transport processes via synergistic activation of the membrane proteins identified.

Thus, characterization of the profile of membrane receptors and ion channels expressed on the cell membranes of CFPAC-1 (ATCC CRL-1918), PANC-1 (ECACC N 87092802) and Calu-3 (ATCC HTB-55) cell lines using radioligand binding methods will be performed as described herein. CFPAC-1 is derived from a human pancreatic ductal adenocarcinoma, and Calu-3 is derived from a human lung adenocarcinoma. Both of these cell lines are known to express the defective allele of CFTR (the ΔF508 mutation). PANC-1 is another pancreatic carcinoma cell line that expresses the wild type CFTR allele (without ΔF508 mutation). The three cell lines will be characterized in approximately 80 different in vitro assays to determine the specific ligand binding activity to identify the membrane receptors and the ion channels that are functionally expressed (Good, 1998). It is noteworthy that we have now already substantially characterized both CFPAC-1 and PANC-1 cells and have partially characterized Calu-3 cells. These results are included in the examples set forth hereinbelow.

The signal transduction “response” (i.e., changes in intracellular levels of cAMP or Ca++) profile relevant to a broad range of G-protein coupled receptors (GPCRs) with these three cell lines using a panel (100-200) of receptor specific agonists will be compiled and analyzed in parallel to the characterization of the receptor profiles on the cell lines described above. A specific set of agonist compounds will be selected from a library of known pharmacological reference agents and other bioactive compounds. They will be selected to cover a diverse array of GPCRs, as well as ion channels. The characterization methods that we will employ will be identical to the assay conditions used in characterizing cellular functional responses to a receptor agonist.

Any potential receptor-mediated synergistic effects between and among the receptors and ion channels will then be characterized. Based on the cellular functional profiles (the types of receptors and types of signal transduction pathways) and the effects of individual receptor ligands, selected combinations of these chemicals (n≧2) will be employed in assays to assess whether there is a combined effect that is more “pronounced” (i.e., enhanced potency) or synergistic and greater than the sum of the responses produced by individual agents.

Potential receptor-mediated individual and combined synergistic effects on cellular chloride efflux will then be compared and characterized. Based on the cell expression profiles (the types of receptors) and the effects of individual receptor ligands, a selected combination of chemicals (n≧2) will be used in the Cl— efflux assay to assess whether there is a combined effect that is more pronounced than that of a single chemical agent (Chapp et al, 2003). Our approach is based partly on the hypothesis that CFTR activation requires phosphorylation at both the NBD binding and R domains. The phosphorylation at the NBD site requires the activation of protein kinase C (PKC), whereas the phosphorylation at the other domain, “R”, requires the activation of protein kinase A (PKA). Using the approaches described above, the impact on Cl— secretion via concurrent or concomitant effects (or stimulation) of the different signal transduction pathways (or the same mechanistic pathway but activation via different receptors) will be determined. In other words, we will determine whether such interacting ligands can compensate for the chloride channel abnormalities associated with CFTR by activating other chloride-related signal transduction pathways that are stimulated by different receptors.

The following examples are provided to illustrate certain embodiments of the invention. They are not intended to limit the invention in any way. The materials and methods are provided to facilitate practice of the present invention.

### EXAMPLE I

Homogenate Preparations

Cells (approx. 1-3×10⁸) were centrifuged (1,000 rpm), homogenized with a Polytron (5×10 sec.) in fresh buffer (Krebs Ringer) with an aliquot taken for protein determination (BioRad Protein Assay Kit). The homogenate was centrifuged (48,000 g) for 10 minutes at 4° C. The pellet was suspended to afford a solution of 4 mg/ml of protein and frozen and stored at −80° C. until use. The protein concentration was determined from the aliquot as indicated to determine the dilution of the pellet to 4 mg/ml.

Assays

All assays follow standard protocols found on the world wide web at novascreen.com, based on modified methods listed in the given references. Essentially, the methods involve initiation of binding by adding the suspended cell pellet preparations (above) to the radioligand for a selected receptor in the presence and absence of non-radiolabeled competitor. Concentrations of the nonlableled competitor were normally 10 μM, sufficient for non-specific binding. Radioligand concentrations were kept at near the KD for specific receptors and were about equivalent to Kᵢs. Binding was carried out for about 2 hrs at room temperature and terminated by filtration, followed by extensive washing and drying at 42° C. Plates were sealed with TopSeal and counted on a Packard Topcount.

Data Handling and Analysis

All data for binding activity were reduced using standard computational programs. The criteria for positive expression of a target protein in a given cell line require that specific binding of the ligand is at least 50% and that this specificity represents at least 300 dpm per 50 μg of cell protein. All data points were performed in triplicate and positive expression of a target protein was reconfirmed in a second assay.

Stepwise Protocol For an Agonist Assay

Changes in intracellular Ca++ concentration when HL-60 cells are exposed to UTP, which is a purinergic P2Y receptor (Gq coupled) agonist (Jin et al, 1998) were measured as follows:

Cell Preparation—Cells were removed from the flask and washed twice with HBSS (1000 RPM for 5 min.). Approximately 5×10⁴ cells in 110 μL of media were added. 70 μL calcium dye (Molecular Devices) was then added. The cells were incubated for one hour at 37° C. and centrifuged at (1000 RPM for 5 min.) to facilitate attachment to the plate.

Agonist-Antagonist-Assay is performed at 37° C.

1. Place plate in fluorometer (FlexStation, Molecular Devices). Excitation/emission is 485/525 with a 515 nm cut-off. Set readings to approximately 2 sec per reading with 30 seconds before addition of first drug.

2. AGONIST: For agonist assay, add 20 μL of UTP or the drug of interest, at 10× the final concentration. Read for an additional 60 seconds.

3. For calibration of the calcium response, add 20 μL of 100 μM A23187 calcium ionophore (dye), and measure increase in fluorescence for one minute. Then add 20 μL 50 mM EGTA, 2M Tris pH 7.4, 0.1% Triton and measure the response until it equilibrates (one minute). The equation [Ca++]=Kd×(F−Fmin)/(Fmax−F) is used to calculate the calcium levels at various fluorescence levels (F). Fmin. is the fluorescence with EGTA and Fmax is the fluorescence with A23187; Kd=370 nM.

Materials

1. 96-well plates: black (sides), clear bottom, sterile, tissue culture treated with lids (VWR #29443-152).

2. Molecular Devices Calcium Dye: Dilute vial of dye from MDC as directed by kit, using HBSS. Further dilute 1:10 in HBSS before use.

3. Cell culture. HL-60 cells. Maintain cells in RPMI-1640 medium, supplemented with FBS 10% (20% for new cultures), 2 mM L-glutamine at 37° C., with 5% CO₂ humidified atmosphere. Cells are non-adherent. Subculture every 3-4 days by seeding at 3×10⁵ cells/mL and grow to a maximum of 2×10⁶ cells/mL. Differentiation toward granulocytes using 1% DMSO was utilized. EGTA: (Ethylene glycol-bis(beta-aminoethyl ether)-N,N,N′,N′-tetraacetic acid) 500 mM in 2M Tris Base. For 10 ml EGTA, add 1.90 g/10 ml 2M Tris Base. Store at −20° C. Add Triton X to a final concentration of 0.01%.

4. A23187 (aka calcimycin), calcium ionophore, MW=523.62 for the free acid. Solubility=5 mg/ml in EtOH, 50 mg/ml in DMSO. Add 0.95 ml DMSO to 1 mg A23187 for a 10 mM stock solution. Store at −20° C.

5. HBSS (HEPES buffered Hank's buffered saline solution). HBSS is NaCl 118 mM (6891 mg/l), KCl 4.6 mM (343 mg/l), CaCl₂ 1 mM (111 mg/L), MgCl₂ 1 mM (95.2 mg/L), glucose 10 mM (1802 mg/L) and HEPES sodium salt 20 mM (5200 mg/L), adjusted to pH 7.2 with HCl.

6. UTP (Agonist) Sigma #U6625 or equivalent, MW=586.1. Store below 0° C. For agonist dose response, test at 0, 1×10 ⁻¹⁰, ×10⁻⁹, ×10⁻⁸, ×10⁻⁷, ×10⁻⁶, ×10⁻⁵, ×10⁻⁴M final concentrations, dissolving in HBSS.

### RESULTS

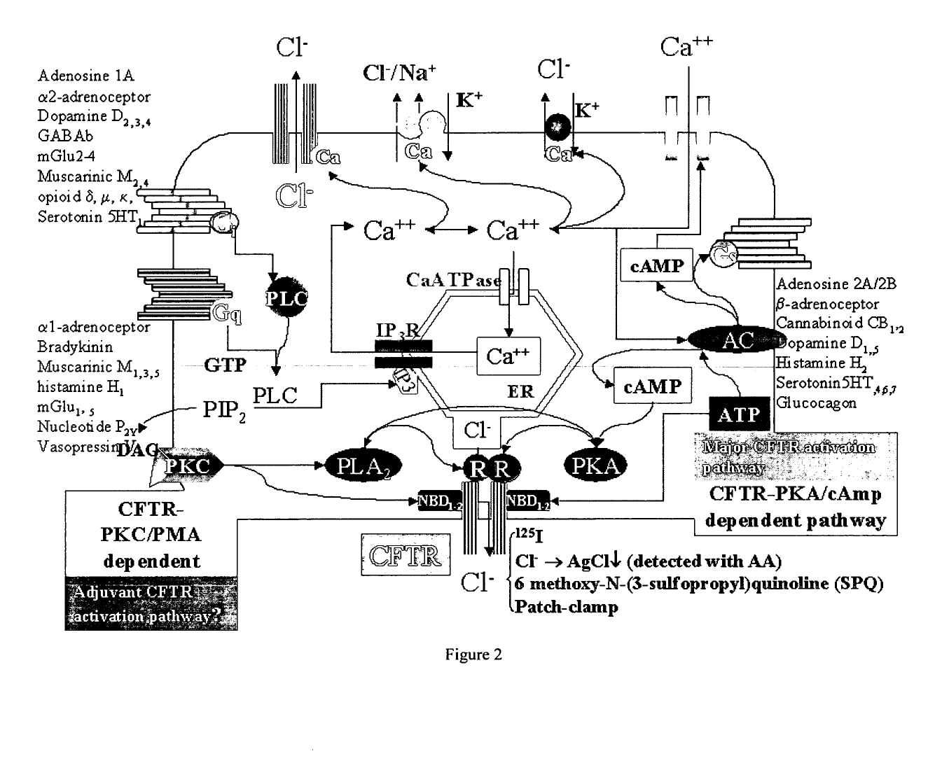

CFTR is a Cl— -channel that mediates cAMP-dependent Cl— efflux in various epithelia. CFTR is activated by the cAMP signal transduction pathway linked to the activity of adenylyl cyclase. G proteins (Gαs/i) mediate the activity of adenylyl cyclase, which in turn attenuate the intracellular concentration of cAMP, hence the activity of CFTR. FIG. 1A shows a diagram depicting the five domains of the CFTR membrane protein. FIG. 1B is a schematic diagram of a G-protein coupled receptor. FIG. 2 presents a number of possible G-protein coupled receptor regulated phosphorylation pathways involved in activation of the wild type CFTR. It is known that stimulation of a Gₛ coupled receptor will induce adenylyl cyclase (AC) activity, resulting in activation of protein kinase A (PKA), and leading to Cl⁻ secretion via CFTR. There is also evidence that the stimulation of G_{q} coupled receptors (such as purinergic P2Y) will induce PKC/PLA2 activity, hence forcing Cl⁻ secretion.

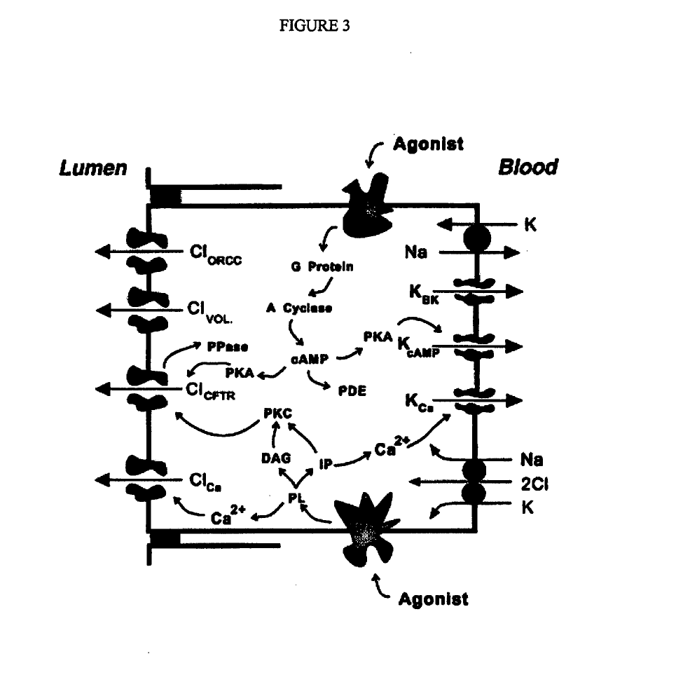

As shown in FIG. 3, there are several Cl⁻—transport mechanisms in epithelial cells other than CFTR; many of which can be affected by the intracellular concentration of [cAMP], or [Ca⁺⁺]. The level of these intracellular secondary messengers may be modulated via the activities of G-protein coupled receptors; for example, stimulation of a G_{q} coupled receptor could result in the release of the stored Ca⁺⁺ from ER (depends on the type of G-protein coupling), leading to a rise in intracellular Ca⁺⁺ concentration. Stimulation of Gₛ coupled receptors promotes the intracellular generation of cAMP. The net results can be, as shown in the Figure, promotion of cellular ion exchange.

A significant number of immortalized cell lines have been characterized for the expression of natively expressed proteins, which can be used for drug screening and discovery. Table 1 summarizes the cell profiling results of two different cell lines, CFPAC-1 and PANC-1. Both of these pancreatic epithelial cell lines exhibited the presence of adrenergic-α1(G_{q}), β-2 (Gₛ), neurotensin (G_{q}/Gᵢ), and purinergic P2Y (G_{q}) receptors (shaded in light blue); some of which were known and previously reported (Nylander and Flemstrom, 1986; de Ondarza and Hootman, 1995; al-Nakkash et al, 1996; Clarke et al, 2000). However, there are differences between the two cell lines. CFPAC-1, originated from a patient with pancreatic cancer with co-morbid cystic fibrosis, i.e. the CFTR is the mutated form. In CFPAC-1, there is apparent up-regulation of several receptors and ion channels including α-2 adrenoceptors (Gi), Angiotensin II receptor type I (AT1) receptors (G_{q}/Gᵢ, Chan et al, 1997), complement C5aR (CD88, Gᵢ), and melatonin (subtype unknown, Gᵢ/G_{q}). Following each receptor named, we indicate the possible G-proteins and the associated detectable cellular functions as changes of intracellular concentration of secondary messengers. That is, both Gs/Gi coupled receptors may be characterized by [cAMP] determinations; whereas G_{q} is characterized by intracellular [Ca⁺⁺] changes.

As previously stated, primary cells express many different types of receptors and ion channels when stimulated with receptor or ion channel specific agonists. Some of these receptors or ion channels will generate responses that can be detected with standard techniques. For example, HL-60 is a human leukemia cell line widely used as a model system for immunological analyses and reportedly expresses complement C5aR, leukotriene B4 (LTB4), and platelet-activating factor (PAF) receptors, receptors for purine and pyrimidine nucleotides, histamine H1-and H2-receptors, β2-adrenoceptors, and prostaglandin receptors (Klinker et al, 1996).

TABLE I
Two cell profiles (PANC-1 and CFPAC-1 cells)
obtained using radioligand-binding methods.

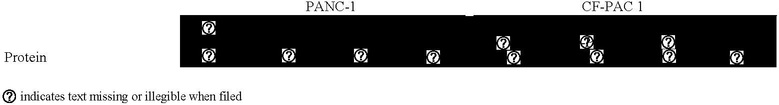

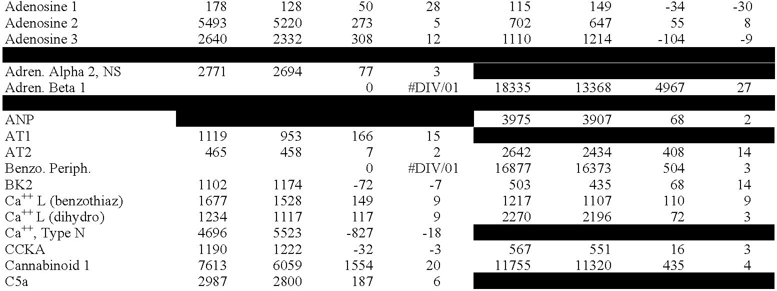

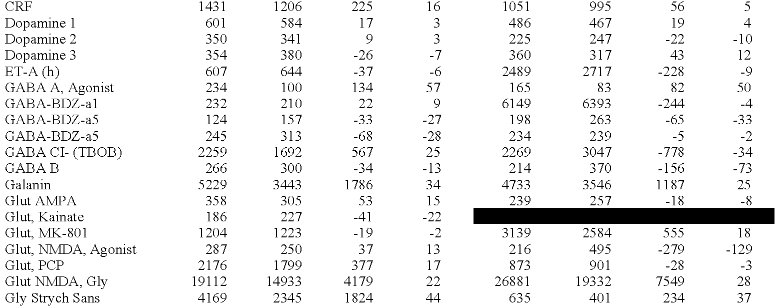

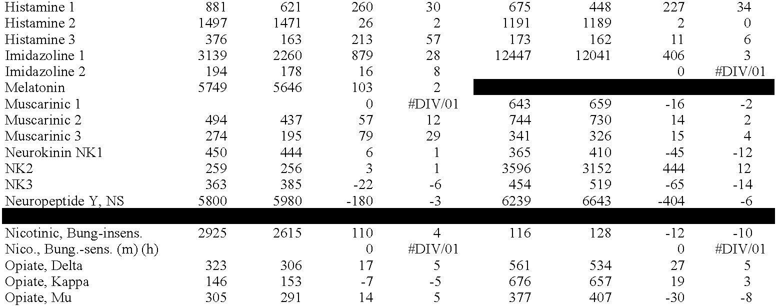

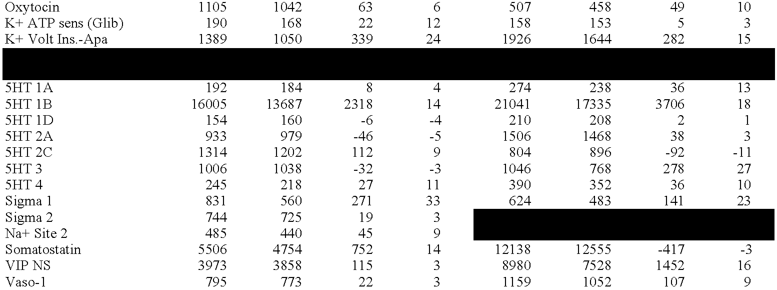

Both methods, either the measurement of cAMP production or intracellular [Ca⁺⁺], use conventional drug screening methodology. Cells may be seeded and prepared in 96-well (or 384-well) plates. When carefully chosen receptor selective/specific agonists are added to each of the wells, there will be a profile of cellular responses, for instance changes of [Ca⁺⁺] or [cAMP]. Those that are registering positive responses to a receptor-selective (-specific) ligand or chemical imply the possible existence of the respective receptor. When using more than one selective ligand as proposed, the other respective ligands selective to the same receptor, when producing similar responses, confirm the presence of a specific receptor.

These approaches, whether binding or functionality based, have some practical utilitarian appeal, which will directly effect medication development. In fact, the results of these studies have revealed some interesting targets and we are now in the process of obtaining the cell profile of Calu-3, a lung derived epithelial cell line. The additional results (both Calu-3 cell receptor profiles and the remaining profiles for CFPAC-1 and PANC-1) will indicate the elements of consensus and of disparity amongst the cells of different tissues of the same disease type (CF). Utilizing this information, we will rationally devise specific drug development strategies to produce more effective treatments for cystic fibrosis.

The three cell lines will be characterized in a rather comprehensive list of 80 different proteins/assays summarized in Table 2, which is comprised of mostly GPCR receptors, some ligand gated ion channels, and ion channels.

TABLE 2
Cell lines and receptors to be assayed
List of Receptors and Ion Channels

Adenisine A1
Adenisine A2
Adenosine A3
Adrenergic alpha 1
Adrenergic alpha 2A
Adrenergic alpha 2B
Adrenergic alpha 2C
Adrenergic beta 2
ANF, atrial natriuretic factor/peptide
angiotensin
AT1, angiotensin 1
AT2, angiotensin 2
Bradykinin, BK2
C5a Complement C5A
Calcium Channel, Type L (benzithiazepine site)
Calcium Channel, Type L (dihydropyridine site)
Calcium Channel, Type N
canabinioid 1
canabinioid 2
CCK1 cholecystokinin
CCK2 cholecystokinin
CRF, corticotropin releasing factor
Dopamine D1
Dopamine D2
Dopamine D3
EGF epidermal growth factor (tyrosine kinase R)
ETA endothelin
GABA A agonist site
GABA A BDZ alpha 1 site
GABA A BDZ alpha 5 site
GABA A BDZ alpha 6 site
GABA B
GABA, Chloride, TBOB site
Galanin
Glutamate, AMPA site
Glutamate, Kainate site
Glutamate, NMDA agonist site
Glutamate, NMDA, glycine (stry-insens site)
glyc, stry-sens
Histamine H1
Histamine H2
Histamine H3
Imidazoline, I1
Imidazoline, I2
Melatonin
Muscarinic M1
Muscarinic M2
Muscarinic M3
Neurokinin, NK1
Neurokinin, NK1
Neurokinin, NK2
Neurokinin, NK3
Neurokinin, NK3
Neuropeptide Y
Neurotensin
Neurotensin
Nicotinic, Bgtx insitive
Nicotinic, Bgtx sens.
Opioid, delta 1
Opioid, kappa 1
Opioid, Mu
oxytocin
PAF, platelet activating factor
Potasium Channel, ATP-sensitive
Potasium Channel, Ca++ Act., VI
Potasium Channel, Ca++ Act., VS
Purinergic, P2Y
Serotonin, 5HT1A
Serotonin, 5HT1B
Serotonin, 5HT1D
Serotonin, 5HT2A
Serotonin, 5HT2C
Serotonin, 5HT3
Serotonin, 5HT4
Sigma 1
Sigma 2
Sodium Site 2
somatostatin
Somatostatin
TRH, thyrotropin releasing hormone
Vasoactive Intestinal Peptide
Vasopressine 1

In summary, we will identify membrane receptors and ion channels on the three cell lines, via detection with radioligand methods. This information will facilitate the determination as to whether there are synergistic or potentiated combinations of targets which may be useful in normalizing Cl— efflux in epithelial cells expressing mutated CFTR.

### EXAMPLE 2

### Activating Other Chloride Channels as CFTR Substitutes

In addition to CFTR, other chloride channels exist in the cell membrane. Conceivably, these other channels could substitute for the defective CFTR protein to prevent the symptoms of CF. The functions of these additional channels and the mechanisms by which they are opened and closed are not well defined. Recent data suggest that CFTR itself may regulate these other channels, in conjunction with factors such as the concentration of calcium ions or the cell volume. Researchers are studying chloride channels and their regulatory mechanisms hoping to learn how to activate these channels and bypass the CFTR defect.

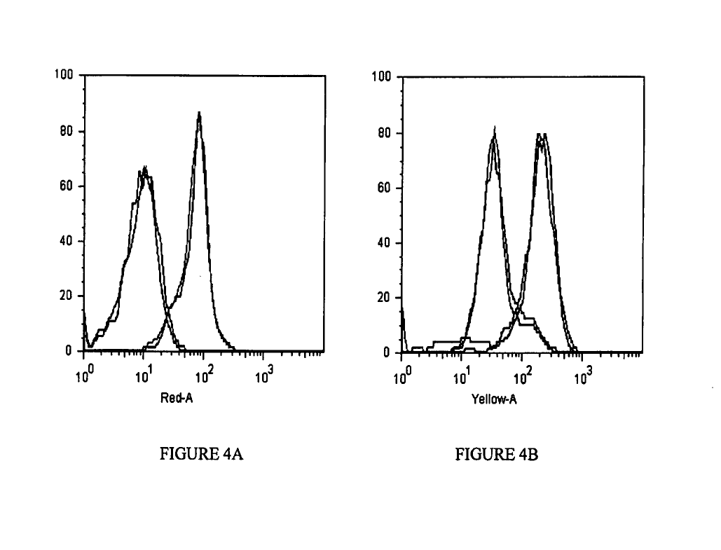

We have begun to characterize the receptor profile on Calu-3 cells. As shown in FIG. 4, FACS analysis has revealed the presence of the CXCR2/CXCR2B (isoforms of the IL-8 receptors) in Calu-3 cells which were derived from a lung adenocarcinoma line. It is known that infections activate innate immunity including the activation of complement pathways and toll-like receptors. Activation of innate immunity releases peptides like C5a (which plays a role in complement pathways) and chemokines such as IL-8 (toll activation). Released C5a and IL-8 activate membrane receptors such as CXCR2 and C5aR (CD88). Activation of CXCR2 and C5aR (both Gαi coupled membrane receptors) inhibits adenylyl cyclase activity. Inhibition of adenylyl cyclase activity inhibits a multitude cellular functions, including cellular ion exchange, more specifically Cl— transport.

G-protein coupled receptors mediate a multitude of cellular functions including ion transport. GPCR may be able to modulate cellular functions of salt (NaCl), fluid transport via alternative mechanisms other than CFTR. GPCRs also mediate cell function including gene expression and protein production. Modulating GPCR activities may reduce (or completely inhibit) cellular production of pro-inflammatroy factors like IL-8. Agents, individually or in combinations, capable of modulating one or more receptor activities are likely helpful in managing and even ameliorating certain conditions associated with cystic fibrosis.

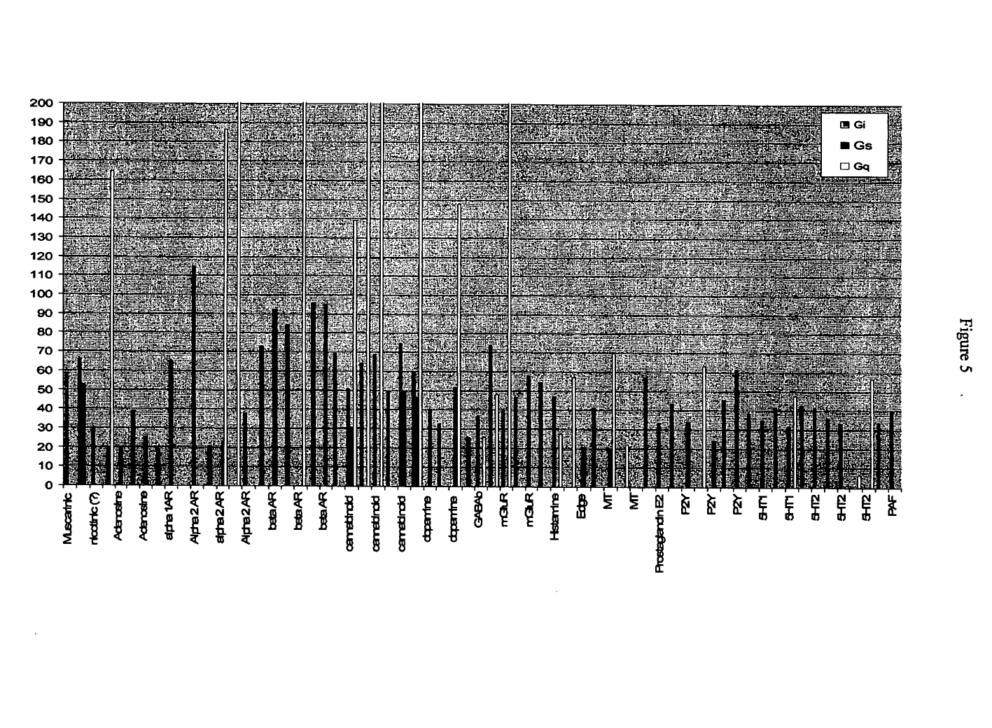

FIG. 5 is a graph showing the Calu-3 GPCR response profile to a collection of 85 small molecule receptor agonists.

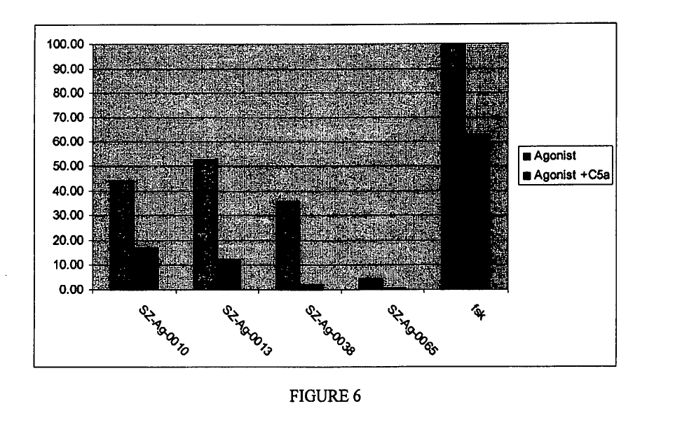

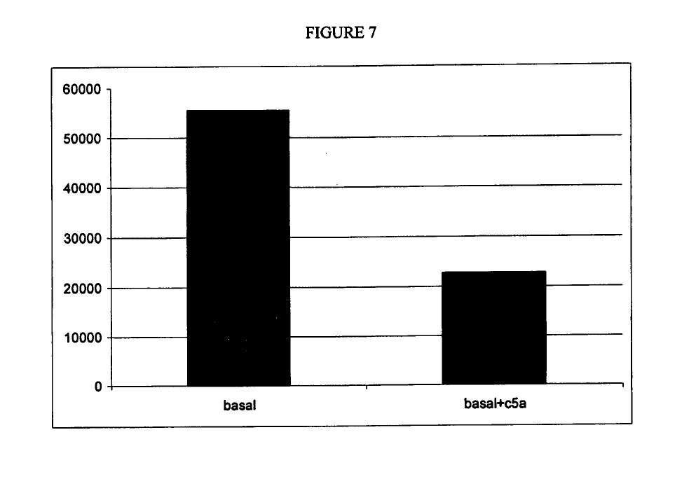

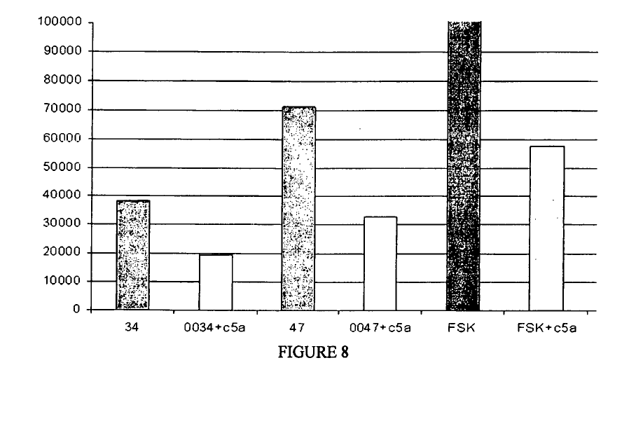

C3a and C5a anaphylatoxins are two proinflammatory peptides generated during complement activation that act through distinct Gαi protein-coupled receptors named C3aR and C5aR, respectively. FIG. 6 is a graph which illustrates that there is GPCR cross talk in Calu-3 cells and further shows that C5a inhibits adenylyl cyclase activity. The data provided in FIG. 7 indicate that C5a inhibits basal Cl— efflux via the C5aR (CD88). C5a also inhibits cAMP dependent Cl— efflux. See FIG. 8.

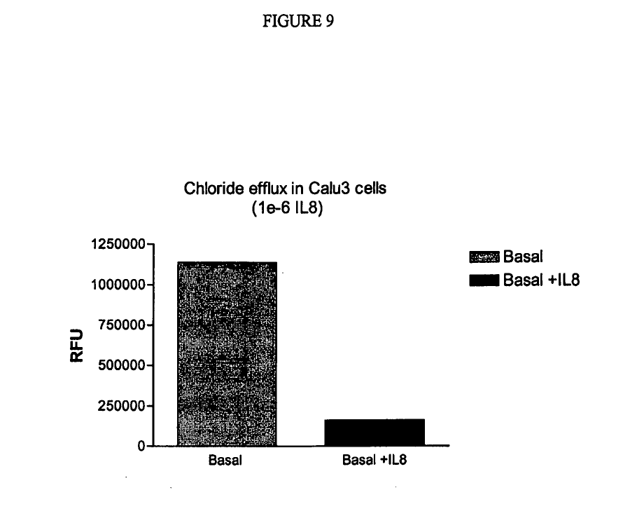

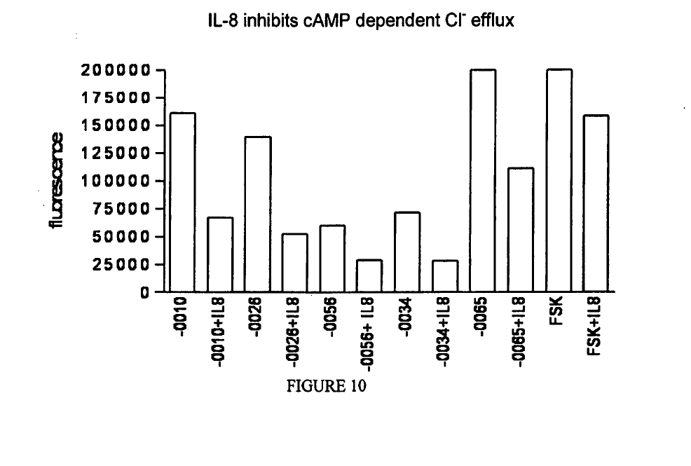

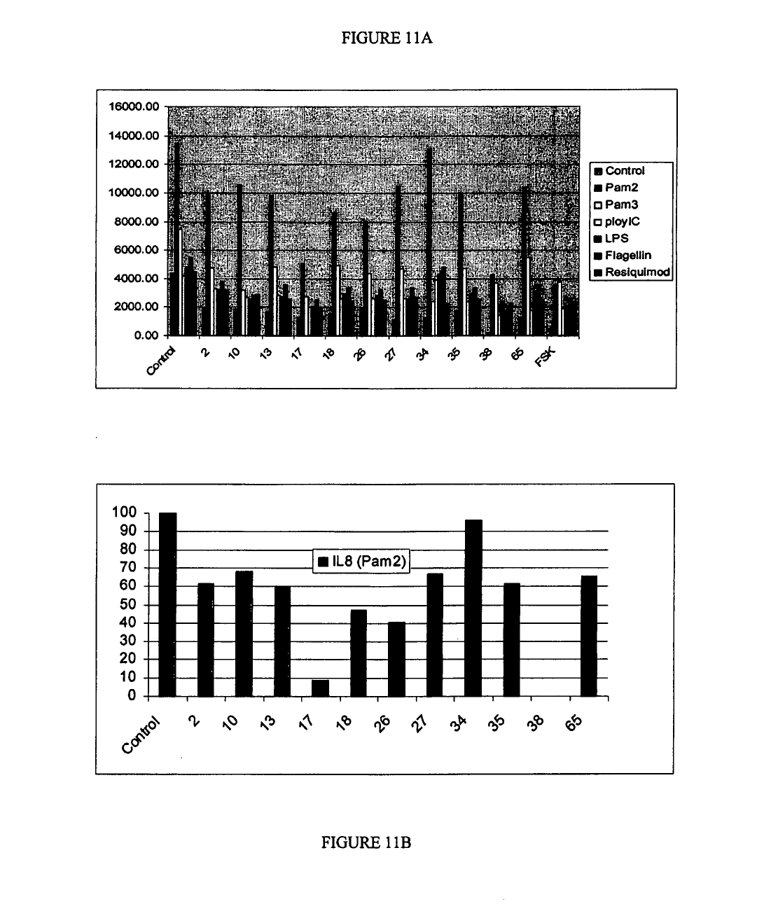

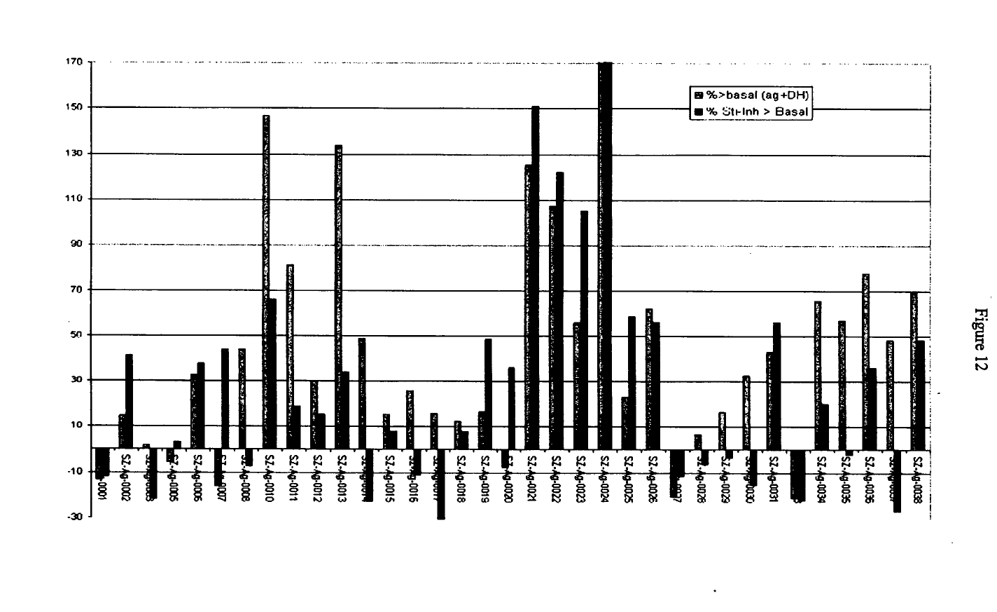

As shown in FIG. 9, IL-8 inhibits basal levels of Cl— transport via chemokine receptors (CXCR2/b). As with C5a, IL-8 also inhibits cAMP dependent Cl— efflux. See FIG. 10. Chemokine/cytokine profiling detected high basal IL-8 “secretion” in Calu-3 cells. Some agonists will reduce IL-8 secretion (when TLR2 and 6 are stimulated) Gsα stimulation may reduce IL-8 production. However, certain receptors appear to be more effective than others. See FIGS. 11A and 11B.

IL-8 and C5a are a key contributing inhibitory factor responsible in deficient ion transport. C5a (IL-8, and other receptor ligands) will trigger proinflammatory chemokine IL-8 production with GPCR signal transduction pathways. Gαₛ-protein coupled receptors may reduce IL-8 secretion, but some only produce limited impacts. Gαs-protein coupled receptor may reduce (basal) IL-8 secretion. Dopamine D1 receptor agonists may also reduce proinflammatory cytokines and chemokines significantly. Some GPCR may produce functional crosstalks to enhance Cl— efflux and reduce IL-8 production. Such “positive” receptor “dialogues” are limited to certain combination of receptors.

### EXAMPLE 3

It appears that concurrent stimulation of Adenosine A2B and dopamine D1 receptors may provide synergistic action promoting ion transport and reduction of proinflammatory cytokines and chemokines. 5′-N-ethylcarboxamido-adenosine (NECA) and dihydrexidine may be an efficacious drug combination for the treatment of cystic fibrosis and other pulmonary inflammatory conditions, like COPD.

Another possible and more standard (less risky) drug combination may be combinations of β2 adrenoceptor agonists with dopamine D1 agonists, like dihydrexidine. Note, the D1 selectivity is important, activity crossover to other dopamine receptors, D2 likes, may render the drug ineffective. There is plenty of experimental evidence generated during the past three decades indicating the interaction of receptors between dopaminergic, adenosine and glutamatergic receptors (Fuxe et al, 2003; Moldrich and Beart, 2003; Swope et al, 1999; Sebastiao and Ribeiro, 1996; Daly 1976). Ferré et al, 1998 described a typical example of Gᵢ (adenosine A1)-Gₛ (dopamine D₁) receptor potentiation upon concurrent exposure of receptor agonist and antagonist. In the presence of a Gᵢ receptor (adenosine A1) agonist, CPA, the EC₅₀ of dopamine (upon D₁) is right-shifted (as compared to controls). That is, dopamine is becoming a less effective D₁ stimulator for the production of cAMP. In contrast, in the presence of adenosine A1 antagonist, DPCPX, the EC₅₀ of dopamine (upon D₁) is left-shifted, indicating that dopamine agonist activity is enhanced in the presence of adenosine A1 receptor antagonist. A similar receptor potentiation between dopamine D₂ (Gi) and adenosine A_{2A} (Gs) receptors has also been reported (Ferré et al, 1992, 1994, 1997).

There are an increasing number of experimental demonstrations that these examples are common rather than idiosyncratic. In addition, there are recent reports on receptor cross-talk between GABA receptors (Marshall, et al, 1999), between EGF and PDGF receptors (Graves et al, 2002), between mu-opioid and chemokine CCR5 receptors (Chen et al, 2004); between opioid receptor subtypes, mu and delta (Gomes et al, 2004), between endothelin A and B receptors (Gregan et al, 2004) and between different adrenoceptors (Hague et al, 2004) and these are just a few most recently published examples. That is, when presented in the same cell, the presence of these receptors and their functions should not be considered statically. They are dynamically interacting with each other, and their cellular responses or functions are often generated as the consequence of these receptor “consortiums”.

Known dopamine D1 Agonists

(Dihydrexidine) (RSM-0002179)

Adrogolide (ABT-43 1, DAS-43 1)

Dinapsoline

A86929

A-77636 (RSM-0000762)

SCH23390 (RSM-0000880)

SKF38393 (RSM-0000220)

SKF81297

SKF82957

SKF82958

SKF82959

### EXAMPLE 4

### The Roles of A2b Receptor in Lung Epithelia

Physical stimulation of airway surfaces evokes liquid secretion, but the events that mediate this vital protective function are not understood. When cystic fibrosis transmembrane conductance regulator (CFTR) channel activity was used as a functional readout, we found signaling elements compartmentalized at both extracellular and intracellular surfaces of the apical cell membrane that activate apical Cl conductance in Calu-3 cells. At the outer surface, ATP was released by physical stimuli, locally converted to adenosine, and sensed by A2B adenosine receptors. These receptors couple to G proteins, adenylyl cyclase, and protein kinase A, at the intracellular face of the apical membrane to activate colocalized CFTR. Thus, airways have evolved highly efficient mechanisms to “flush” noxious stimuli from airway surfaces by selective activation of apical membrane signal transduction and effector systems.

There are a number of experimental observations that xanthines, such as IBMX, are able to stimulate some activity even in the mutant forms (ΔF508) of CFTR (Yang et al, 1993; Haws et al, 1996) to partially restore the chloride transport function. However, there are different opinions about the mechanisms of action of this effect. One opinion is that adenosine A1 (a Gᵢ-coupled membrane receptor) is involved mechanistically in restoring the functions of the ion channel (Pereira et al, 1998). Considering that the majority of CFTR protein never reaches the apical membrane, exploration of the other ion channel activities (that control the overall cellular secretion of chloride) may be more prudent and fruitful.

Another example belonging to the same family of xanthines is CPX, or 8-cyclo-pentyl-1,3-dipropylxanthine. This compound may have a different mechanism in promoting halide efflux (Eidelman et al, 1992; Guay-Broder et al, 1995; Jacobson et al, 1995) than that of IBMX. There are different reports of whether this compound functions directly via CFTR.

Flavonol, or quercetin, is known (or observed) to induce Cl⁻ secretion in rat colon epithelial cells. This is at least in part attributed to the activation of baso-lateral K+ channel activities (Cermak et al, 2002).

Regulation of normal CFTR activity is coordinated between the nucleotide binding domains (NBD) that hydrolyze ATP and the phosphorylation/de-phosphorylation at the R-domain by different cellular kinases, phosphatases and phospholipases. In coordination with the NBD, the “opening” of the wild type CFTR is mainly regulated by cAMP-dependent PKA activity. In mutations, this pathway is no longer functional or only partially functional. However, additional kinases (PKC for example) and phospholipases (PLA2 for example) also play a role in CFTR regulation (Chapp et al, 2003; Devor and Pilewski, 1999). There are some examples that the defective ion transport activity may be restored (perhaps only partially) via the activation of pathways other than that of cAMP-dependent PKA activity (Cobb, 2002; Berguerand et al, 1997).

FIG. 12 illustrates that stimulation of the identified receptors can exhibit synergistic effects on cAMP activation and Cl— efflux. Advances in recent years have provided ample evidence that co-expressed membrane proteins are not entirely “independent” of each other in modulating cellular functions. For instance, in macrophages, adenosine 2A receptor (A_{2A}R, Gs) agonists synergize with E. coli LPS (bacterial endotoxin) via the toll-like receptor, TLR4, to up-regulate vascular endothelial growth factor (VEGF) expression in murine macrophages (Leibovich et al, 2002). Moreover, stimulation of other TLRs, TLR2, TLR7, and TLR9, with agonists (different from bacterial endotoxin) “also synergizes with A_{2A} receptor agonists and adenosine to up-regulate VEGF, while simultaneously strongly down-regulating TNF-alpha expression” (Pinhal-Enfield et al, 2003). Thus, exemplary membrane protein based approaches (Cl-e↑/IL-8↓) could entail the following method steps.
Transform Calu-3 (WT-CFTR) and another lung epithelial cell line (ΔF508) with -Luc to make CREB (cAMP response element-binding)-Luc (MM), surrogate cAMP fluorescent reporter;
Select diverse collections of chemical libraries (˜200K compounds; subtract undesired effects, e.g. α1, α2, β2 adrenoceptor, etc);
Screen library against Calu-3-Luc, identify hits;
Second screen against ΔF508-cell-Luc, identify secondary hits;
Characterize effect of hits on Cl— efflux/IL-8 secretion;
Characterize for Pharmacological (receptor, safety and side effects) safety profiles;
Medicinal Chemistry iterations;
In vivo safety and pharmacokinetic assessment.
   Pathway dependent approach (IL-8↓)
Identify key kinase(s) pivotal in IL-8 synthesis in lung epithelial cells (e.g. Calu-3, etc) using array technology;
Identify (or contract to) commercial source of desired kinase;
Collect diverse chemical library as indicated previously;
Screen library for hit identification, kinase inhibitors;
Characterize “in vitro efficacy” of the hits using cell based assay for inhibiting IL-8 synthesis and secretion;
Kinase profiling for selectivity;
Characterize for Pharmacological (receptor, safety and side effects) safety profiles;
Medicinal chemistry iterations;
In vivo safety and pharmacokinetic assessment.

We anticipate that synergistic receptor actions will influence cellular ion exchange as a measurable difference in Cl⁻ secretion. As noted above, examples of synergistic actions and enhanced cellular activities between G_{q} coupled receptors (CCK for instance), and between Gₛ and Gᵢ coupled receptors (dopamine and adenosine for example) have been previously described. In light of cellular ion exchange regulated through CFTR, the synergistic action may also be seen between receptors coupled to G-proteins affecting PKC (G_{q} coupled receptors) and PKA (G_{s/i} coupled receptors via cAMP) activities, or combinations of other receptors as well. For instance, it is known that the CFTR is a cAMP-dependent chloride channel; stimulation of G-protein coupled receptors, such as β2-adrenoceptor (Gₛ-coupled, cAMP-upon stimulation) will activate CFTR Cl⁻ efflux.

There is increasing evidence that CFTR activities may also be regulated through synergistic receptor actions. There is a recent report indicating that the modulation of CFTR activities in smooth muscle cells occurs via the coordinated action of β2 adrenoceptors and VIP receptors (Robert et al, 2004). There are also reports indicating that activation of CFTR Cl⁻ channels in mouse heart are coupled to G-protein coupled P2Y purinergic receptors, i.e. a family of G_{q} coupled receptors (Yamamoto-Mizuma et al, 2004). In fact, many of the experimental observations have suggested that CFTR Cl⁻ channel activity is modulated by the basal phosphorylated states of different types of kinases linked to different signal transduction pathways (Nisato et al, 2004; Himmel and Nagel, 2004; Yamazaki and Kitamura, 2003). Different combinations of receptor agonist/antagonist will be used to characterize their combined effect on total Cl⁻ secretion.

Essentially, we will assemble an “inventory” of the homeostasis of epithelial cells (especially those cells expressing the defective mutant CFTR) and the ion exchange effected by the receptor activities. The product of the survey is a composite of radioligand binding profiles and cell responses to chemical stimuli measured in intracellular Ca++/cAMP concentration changes and Cl— efflux. This body of knowledge will then used to identify optimal combinations of compounds (and potential therapeutic targets) to stimulate chloride secretion, thereby providing insight into potentially synergistic pathways toward which new and more effective medications may be developed in treating not only cystic fibrosis, but other related diseases as well. Research efforts to date have yielded little success with respect to use of Cl— channel openers, or efforts to correct the function of mutated CFTR alleles. In contrast, a great deal is known about modulators of membrane receptors and ion channels that can affect ion exchange by cells. This knowledge can be used to advantage in the development of CFTR medications.

### REFERENCES

al-Nakkash L, Simmons N L, Lingard J M, Argent B E Adenylate cyclase activity in human pancreatic adenocarcinoma cell lines. Int J Pancreatol. February 1996;19(1):39-47.
Andersson C, Servetnyk Z, Roomans G M. (2003) Activation of CFTR by genistein in human airway epithelial cell lines. Biochem Biophys Res Commun. 308:518.
Bhatia M, Moochhala S. Role of inflammatory mediators in the pathophysiology of acute respiratory distress syndrome. J Pathol. February 2004;202(2):145-56.
Berguerand M, Klapisz E, Thomas G, Humbert L, Jouniaux A M, Olivier J L, Bereziat G, Masliah J. (1997) Differential stimulation of cytosolic phospholipase A2 by bradykinin in human cystic fibrosis cell lines. Am J Respir Cell Mol Biol. 17(4):481.
Brown M. (2002) Gene therapy trials for cystic fibrosis. Drug Discov Today. 1;7:788.
Bubenik G A. Localization, physiological significance and possible clinical implication of gastrointestinal melatonin. Biol Signals Recept. November-December 2001;10(6):350-66.
Carew M A, Yang X, Schultz C, Shears S B myo-inositol 3,4,5,6-tetrakisphosphate inhibits an apical calcium-activated chloride conductance in polarized monolayers of a cystic fibrosis cell line. J Biol Chem. Sep. 1, 2000;275(35):26906-13.
Chan H C, Law S H, Leung P S, Fu L X, Wong P Y. Angiotensin II receptor type I-regulated anion secretion in cystic fibrosis pancreatic duct cells. J Membr Biol. Apr. 1, 1997;156(3):241-9.
Check E. (2003) Harmful potential of viral vectors fuels doubts over gene therapy. Nature. 423:573.
Cheng Z J, Harikumar K G, Holicky E L, Miller L J. Heterodimerization of type A and B cholecystokinin receptors enhance signaling and promote cell growth. Biol Chem. Dec. 26, 2003;278(52):52972-9.
Cheng Z J, Miller L J. Agonist-dependent dissociation of oligomeric complexes of G protein-coupled cholecystokinin receptors demonstrated in living cells using bioluminescence resonance energy transfer. J Biol Chem. Dec. 21, 2001;276(51):48040-7.
Cermak R, Kuhn G, Wolffram S. (2002) The flavonol quercetin activates basolateral K(+) channels in rat distal colon epithelium. Br. J. Pharmacol. 135:1183.
Chappe V, Hinkson D A, Zhu T, Chang X B, Riordan J R, Hanrahan J W. (2003) Phosphorylation of protein kinase C sites in NBD1 and the R domain control CFTR channel activation by PKA. J Physiol. 548(Pt 1):39-52.
Clarke L L, Harline M C, Gawenis L R, Walker N M, Turner J T, Weisman G A. Extracellular UTP stimulates electrogenic bicarbonate secretion across CFTR knockout gallbladder epithelium. Am J Physiol Gastrointest Liver Physiol. July 2000;279(1):G132-8.
Cobb B R, Ruiz F, King C M, Fortenberry J, Greer H, Kovacs T, Sorscher E J, Clancy J P. (2002) A(2) adenosine receptors regulate CFTR through PKA and PLA(2). Am J Physiol Lung Cell Mol Physiol. 282(1):L12-25.
Daly J W. The nature of receptors regulating the formation of cyclic AMP in brain tissue. Life Sci. Jun. 15, 1976;18(12):1349-58.
Dawson D C, Smith S S, Mansoura M K. (1999) CFTR: mechanism of anion conduction. Physiol Rev. (1 Suppl):S47-75.
Delagrange P, Jockers R. Melatonin and energy homeostasis: peripheral versus central regulation. Minerva Endocrinol. December 2003;28(4):313-20.
Denning G M, Ostedgaard L S, Welsh M J. (1992) Abnormal localization of cystic fibrosis transmembrane conductance regulator in primary cultures of cystic fibrosis airway epithelia. J Cell Biol. 118:551.
de Ondarza J, Hootman S R. Regulation of cyclic AMP levels in guinea pig pancreatic ducts and cultured duct epithelial monolayers. Pancreas. October 1995;11(3):261-70.
Devor D C, Pilewski J M. (1999) UTP inhibits Na+absorption in wild-type and DeltaF508 CFTR-expressing human bronchial epithelia. Am J Physiol. (4 Pt 1):C827
Drouin S M, Kildsgaard J, Haviland J, Zabner J, Jia H P, McCray P B Jr, Tack B F, Wetsel R A. Expression of the complement anaphylatoxin C3a and C5a receptors on bronchial epithelial and smooth muscle cells in models of sepsis and asthma. J Immmunol. Feb. 1, 2001;166(3):2025-32.
Duszyk M, MacVinish L, Cuthbert A W. (2001) Phenanthrolines—a new class of CFTR chloride channel openers. Br. J. Pharmacol. 134(4):853.
Eidelman O, Guay-Broder C, van Galen P J, Jacobson K A, Fox C, Turner R J, Cabantchik Z I, Pollard H B. (1992) A1 adenosine-receptor antagonists activate chloride efflux from cystic fibrosis cells. Proc Nati Acad Sci USA. 89:5562.
Feoktistov I, Goldstein A E, Biaggioni I. (1999) Role of p38 mitogen-activated protein kinase and extracellular signal-regulated protein kinase in adenosine A2B receptor-mediated interleukin-8 production in human mast cells. Mol. Pharmacol. 55:726.
Ferre S, Fuxe K, von Euler G, Johansson B, Fredholm B B. Adenosine-dopamine interactions in the brain. Neuroscience. December 1992;51(3):501-12.
Ferre S, Fredholm B B, Morelli M, Popoli P, Fuxe K. Adenosine-dopamine receptor-receptor interactions as an integrative mechanism in the basal ganglia. Trends Neurosci. October 1997;20(10):482-7.
Ferre S, Schwarcz R, Li X M, Snaprud P, Ogren S O, Fuxe K. Chronic haloperidol treatment leads to an increase in the intramembrane interaction between adenosine A2 and dopamine D2 receptors in the neostriatum. Psychopharmacology (Berl). November 1994;116(3):279-84.
Ferre S, Torvinen M, Antoniou K, Irenius E, Civelli O, Arenas E, Fredholm B B, Fuxe K. (1998) Adenosine A1 receptor-mediated modulation of dopamine D1 receptors in stably cotransfected fibroblast cells. J Biol Chem. 273:4718.
Frings S, Reuter D, Kleene S J. Neuronal Ca2+-activated Cl— channels—homing in on an elusive channel species. Prog Neurobiol. February 2000;60(3):247-89.
Fuxe K, Agnati L F, Jacobsen K, Hillion J, Canals M, Torvinen M, Tinner-Staines B, Staines W, Rosin D, Terasmaa A, Popoli P, Leo G, Vergoni V, Lluis C, Ciruela F, Franco R, Ferre S. Receptor heteromerization in adenosine A2A receptor signaling: relevance for striatal function and Parkinson's disease. Neurology. Dec. 9, 2003;61(11 Suppl 6):S19-23.
Fuxe K, Ferre S, Zoli M, Agnati L F. (1998) Integrated events in central dopamine transmission as analyzed at multiple levels. Evidence for intramembrane adenosine A2A/dopamine D2 and adenosine A1/dopamine D1 receptor interactions in the basal ganglia. Brain Res Brain Res Rev. 6(2-3):258
Gadsby D C, Nairn A C. (1999) Control of CFTR channel gating by phosphorylation and nucleotide hydrolysis. Physiol Rev. 79(1 Suppl):S77.
Gallinaro R, Cheadle W G, Applegate K, Polk H C Jr. The role of the complement system in trauma and infection. Surg Gynecol Obstet. May 1992;174(5):435-40.
Gelman, M., and Kopito R., (2002) Rescuing protein confirmation: -prospects for pharmacological therapy in cystic fibrosis. J. clini. Investi. 110:1591.
Gomes I, Gupta A, Filipovska J, Szeto H H, Pintar J E, Devi L A. A role for heterodimerization of mu and delta opiate receptors in enhancing morphine analgesia. Proc Natl Acad Sci USA. Apr. 6, 2004;101(14):5135-9.
Good, E., Perschke, S, Lopez, R, Chang, S, Kinsler, A., Snowman, A., LaCombe, J., Fedock, M., Zeppetello, R., Zysk, J., (1998) Profiling Established Cell lines as a Means to Screening Diversity. J. Biomol. Screen. 3:231.
Graves L M, Han J, Earp H S 3rd. Transactivation of the EGF Receptor: Is the PDGF Receptor an Unexpected Accomplice? Mol Intervent. July 2002;2(4):208-12.
Gregan B, Jurgensen J, Papsdorf G, Furkert J, Schaefer M, Beyermann M, Rosenthal W, Oksche A. Ligand-dependent differences in the internalization of endothelin A and endothelin B receptor heterodimers. J Biol Chem. Apr. 9, 2004 [Epub ahead of print]
Griesenbach U, Geddes D M, Alton E W. (2003) Update on gene therapy for cystic fibrosis. Curr Opin Mol Ther. 5:489-94.
Griesenbach U, Ferrari S, Geddes DM, Alton E W. (2002) Gene therapy progress and prospects: cystic fibrosis. Gene Ther. 9:1344.
Guay-Broder C, Jacobson K A, Barnoy S, Cabantchik Z I, Guggino W B, Zeitlin P L, Turner R J, Vergara L, Eidelman O, Pollard H B. (1995) A1 receptor antagonist 8-cyclopentyl-1,3-dipropylxanthine selectively activates chloride efflux from human epithelial and mouse fibroblast cell lines expressing the cystic fibrosis transmembrane regulator delta F508 mutation. Biochemistry. 34: 9079.
Hague C, Uberti M A, Chen Z, Hall R A, Minneman K P. Cell surface expression of alphal D-adrenergic receptors is controlled by heterodimerization with alphal B-adrenergic receptors. J Biol Chem. Apr. 9, 2004;279(15):15541-9.
Haws C, Finkbeiner W E, Widdicombe J H, Wine J J. (1994) CFTR in Calu-3 human airway cells: channel properties and role in cAMP-activated Cl— conductance. Am. J. Physiol. 266(5 Pt 1):L502.
Hazinski M. F., Iberti T. J., Maclntyre N. R., Parker M. M., Tribett D, Prion S, Chmel H. Epidemiology, pathophysiology and clinical presentation of gram-negative sepsis. Am J Crit Care. May 1993;2(3):224-35; quiz 236-7.
Himmel B, Nagel G. Protein kinase-independent activation of CFTR by phosphatidylinositol phosphates. EMBO Rep. January 2004;5(1):85-90.
Ho M W, Kaetzel M A, Armstrong D L, Shears S B. Regulation of a human chloride channel. a paradigm for integrating input from calcium, type ii calmodulin-dependent protein kinase, and inositol 3,4,5,6-tetrakisphosphate. J Biol Chem. Jun. 1, 2001;276(22):18673-80. Epub Mar. 5, 2001.
Holz G G. Epac: A new cAMP-binding protein in support of glucagon-like peptide-1 receptor-mediated signal transduction in the pancreatic beta-cell. Diabetes. January 2004;53(1):5-13.
Hwang T H, Schwiebert E M, Guggino W B. (1996) Apical and basolateral ATP stimulates tracheal epithelial chloride secretion via multiple purinergic receptors. Am J Physiol. 270:C1611
Jacobson K A, Guay-Broder C, van Galen P J, Gallo-Rodriguez C, Melman N, Jacobson M A, Eidelman O, Pollard H B. (1995) Stimulation by alkylxanthines of chloride efflux in CFPAC-1 cells does not involve A1 adenosine receptors. Biochemistry. 34:9088.
Jaworek J, Bonio J, Leja-Szpa A, Nawrot K, Tomaszewska M R, Stachura J, Pawlik W W, Konturek S J. Sensory nerves in central and peripheral control of pancreatic integrity by leptin and melatonin. J Physiol Pharmacol. March 2002;53(1):51-74.
Jensen B, Hertz M, Christophersen P, Madsen L. The Ca(2+)-activated K(+) channel of intermediate conductance:a possible target for immune suppression. Expert Opin Ther Targets. December 2002;6(6):623-636
Kaneko S, Yada N, Fukuda K, Kikuwaka M, Akaike A, Satoh M. (1997) Inhibition of Ca2+ channel current by mu- and kappa-opioid receptors coexpressed in Xenopus oocytes: desensitization dependence on Ca2+ channel alpha 1 subunits. Br J Pharmacol. 121(4):806.
Kasai Haruo (1991) Tonic inhibition and rebound facilitation of a neuronal calcium channel by a GTP-binding protein. Proc. Natl. Acad. Sci. USA, 88:8885
Kiehn J, Karle C, Thomas D, Yao X, Brachmann J, Kubler W. (1998) HERG potassium channel activation is shifted by phorbol esters via protein kinase A-dependent pathways. J Biol Chem. 273:25285.
Klinker J F, Wenzel-Seifert K, Seifert R. (1996) G-protein-coupled receptors in HL-60 human leukemia cells. Gen Pharmacol. 1:33.
Kulich M, Rosenfeld M, Goss C H, Wilmott R. (2003) Improved survival among young patients with cystic fibrosis. J Pediatr. 142:631.
Leibovich S J, Chen J F, Pinhal-Enfield G, Belem P C, Elson G, Rosania A, Ramanathan M, Montesinos C, Jacobson M, Schwarzschild M A, Fink J S, Cronstein B. Synergistic up-regulation of vascular endothelial growth factor expression in murine macrophages by adenosine A(2A) receptor agonists and endotoxin. Am J Pathol. June 2002;160(6):2231-44.
Linden J, Thai T, Figler H, Jin X, Robeva A S. (1999) Characterization of human A(2B) adenosine receptors: radioligand binding, western blotting, and coupling to G(q) in human embryonic kidney 293 cells and HMC-1 mast cells. Mol Pharmacol. 56:705.
Liu J, Evans M S, Lee T J. (2002) Presynaptic muscarinic M(2)-receptor-mediated inhibition of N-type Ca(2+) channels in cultured sphenopalatine ganglion: direct evidence for acetylcholine inhibition of cerebral nitrergic neurogenic vasodilation. J Pharmacol Exp Ther. 302:397.
Liu X S, Xu Y J, Zhang Z X, Li C Q, Yang D L, Zhang N, Ni W. Isoprenaline and aminophylline relax bronchial smooth muscle by cAMP-induced stimulation of large-conductance Ca2+-activated K+ channels. Acta Pharmacol Sin. May 2003;24(5):408-14.
Marshall F H, White J, Main M, Green A, Wise A. GABA(B) receptors function as heterodimers. Biochem Soc Trans. August 1999;27(4):530-5.
McCarty N A, Standaert T A, Teresi M, Tuthill C, Launspach J, Kelley T J, Milgram L J, Hilliard K A, Regelmann W E, Weatherly M R, Aitken M L, Konstan M W, Ahrens R C. (2002) A phase I randomized, multicenter trial of CPX in adult subjects with mild cystic fibrosis. Pediatr. Pulmonol. 33:90.
Meyer U A. (2000) Pharmacogenetics and adverse drug reactions. Lancet. 356(9242):1667.
Moldrich R X, Beart P M. Emerging signalling and protein interactions mediated via metabotropic glutamate receptors. Curr Drug Target CNS Neurol Disord. April 2003;2(2):109-22.
Mondino B J. Inflammatory diseases of the peripheral cornea. Ophthalmology. April 1988;95(4):463-72.
Moon S, Singh M, Krouse M E, Wine J J. (1997) Calcium-stimulated Cl— secretion in Calu-3 human airway cells requires CFTR. Am. J. Physiol. 273(6 Pt 1):L1208.
Nelson C S, Marino J L, Allen C N. Melatonin receptor potentiation of cyclic AMP and the cystic fibrosis transmembrane conductance regulator ion channel. J Pineal Res. March 1999;26(2):113-21.
Niisato N, Nishino H, Nishio K, Marunaka Y. Cross talk of cAMP and flavone in regulation of cystic fibrosis transmembrane conductance regulator (CFTR) Cl— channel and Na+/K+/2Cl— cotransporter in renal epithelial A6 cells. Biochem Pharmacol. Feb. 15, 2004;67(4):795-801.
Nylander O, Flemstrom G. Effects of alpha-adrenoceptor agonists and antagonists on duodenal surface epithelial HCO3-secretion in the rat in vivo. Acta Physiol Scand. March 1986;126(3):433-41.
Pereira M M, Lloyd Mills C, Dormer R L, McPherson M A. Actions of adenosine A1 and A2 receptor antagonists on CFTR antibody-inhibited beta-adrenergic mucin secretion response. (1998) Br. J. Pharmacol. 125:697.
Pinhal-Enfield G, Ramanathan M, Hasko G, Vogel S N, Salzman A L, Boons G J, Leibovich S J. An angiogenic switch in macrophages involving synergy between Toll-like receptors 2, 4, 7, and 9 and adenosine A(2A) receptors. Am J Pathol. August 2003;163(2):711-21.
Riedemann N C, Guo R F, Ward P A. A key role of C5a/C5aR activation for the development of sepsis. J Leukoc Biol. December 2003;74(6):966-70.
Robert R, Thoreau V, Norez C, Cantereau A, Kitzis A, Mettey Y, Rogier C, Becq F. Regulation of the Cystic Fibrosis Transmembrane Conductance Regulator Channel by {beta}-Adrenergic Agonists and Vasoactive Intestinal Peptide in Rat Smooth Muscle Cells and Its Role in Vasorelaxation. J Biol Chem. May 14, 2004;279(20):21160-21168.
Sebastiao A M, Ribeiro J A. Adenosine A2 receptor-mediated excitatory actions on the nervous system. Prog Neurobiol. February 1996;48(3): 167-89.
Schultz B D, Singh A K, Devor D C, Bridges R J. (1999) Pharmacology of CFTR chloride channel activity. Physiol. Rev. 79:S109.
Sheppard D N, Welsh M J. (1999) Structure and function of the CFTR chloride channel. Physiol Rev. 79(1 Suppl):S23-45.
Shen B Q, Finkbeiner W E, Wine J J, Mrsny R J, Widdicombe J H. (1994) Calu-3: a human airway epithelial cell line that shows cAMP-dependent Cl— secretion. Am J Physiol. 266(5 Pt 1):L493.
Shimizu T, Morishima S, Okada Y. (2000) Ca2+-sensing receptor-mediated regulation of volume-sensitive Cl— channels in human epithelial cells. J Physiol. 528(Pt 3):457-72
Sorensen J B, Nielsen M S, Gudme C N, Larsen E H, Nielsen R. (2001) Maxi K+ channels co-localised with CFTR in the apical membrane of an exocrine gland acinus: possible involvement in secretion. Pflugers Arch. 442(1):1-11
Srivastava M, Eidelman O, Pollard H B. (1999) Pharmacogenomics of the cystic fibrosis transmembrane conductance regulator (CFTR) and the cystic fibrosis drug CPX using genome microarray analysis. Mol. Med. 5:753
Swope S L, Moss S J, Raymond L A, Huganir R L. Regulation of ligand-gated ion channels by protein phosphorylation. Adv Second Messenger Phosphoprotein Res. 1999;33:49-78.
Thiagarajah J R, Song Y, Haggie P M, Verkman A S. A small molecule CFTR inhibitor produces cystic fibrosis-like submucosal gland fluid secretions in normal airways. FASEB J. May 2004;18(7):875-7. Epub Mar. 4, 2004.
Vajanaphanich M, Schultz C, Rudolf M T, Wasserman M, Enyedi P, Craxton A, Shears S B, Tsien R Y, Barrett K E, Traynor-Kaplan A. Long-term uncoupling of chloride secretion from intracellular calcium levels by Ins(3,4,5,6)P4. Nature. Oct. 20, 1994;371(6499):711-4.
Wagner J G, Roth R A. Neutrophil migration mechanisms, with an emphasis on the pulmonary vasculature. Pharmacol Rev. September 2000;52(3):349-74.
Ward P A, Kunkel S L. Bacterial virulence and the inflammatory system. Rev Infect Dis. September-October 1983;5 Suppl 4:S793-6.
Weddle D L, Tithoff P, Williams M, Schuller H M. Beta-adrenergic growth regulation of human cancer cell lines derived from pancreatic ductal carcinomas. Carcinogenesis. March 2001;22(3):473-9.
Werry T D, Wilkinson G F, Willars GB. Mechanisms of cross-talk between G-protein-coupled receptors resulting in enhanced release of intracellular Ca2+. Biochem J. Sep. 1, 2003;374(Pt 2):281-96.
West M R, Molloy C R. (1996) A microplate assay measuring chloride ion channel activity. Anal Biochem. 241:51.
Yamamoto-Mizuma S, Wang G X, Hume J R. P2Y purinergic receptor regulation of CFTR chloride channels in mouse cardiac myocytes. J Physiol. May 1, 2004;556(Pt 3):727-37.
Yamazaki J, Kitamura K. Regulation of the drug-sensitivity of anion channels via phosphorylation Nippon Yakurigaku Zasshi. November 2003;122 Suppl:67P-70P
Yang Y, Devor D C, Engelhardt J F, Ernst S A, Strong T V, Collins F S, Cohn J A, Frizzell R A, Wilson J M. (1993) Molecular basis of defective anion transport in L cells expressing recombinant forms of CFTR. Hum. Mol. Genet. 2:1253.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for identifying agents which compensate for the defective ion transport of the mutated CFTR in a cell, comprising,
a) providing a plurality of cell lines, at least one cell line comprising a mutation in the CFTR gene and least one cell line being wild type for CFTR expression;
b) contacting said cell lines with at least one agent which modulates intracellular messenger activity, and
c) assessing said cell lines of step a) for an alteration in chloride secretion following incubation in the presence of said agent, agents which increase said chloride ion transport being effective for the treatment of cystic fibrosis.

2. The method of claim 1, wherein said agent is modulates at least one G-protein coupled receptor.

3. The method of claim 1, wherein said agent alters intracellular levels of cAMP or Ca++

4. The method of claim 1, wherein said agent modulates ion channel activity.

5. The method of claim 1, wherein said cell lines are contacted with two agents simultaneously and said cells are assessed to determine if said agents act synergistically to increase chloride ion transport.

6. The method of claim 1, wherein said agent is at least one agent listed in Table 1.

7. The method of claim 1, wherein said agent has binding affinity for a receptor listed in Table 2.

8. The method of claim 1, wherein said agent has binding affinity for an ion channel listed in Table 2.

9. A combination of agents identified by the method of claim 5.

10. A pharmaceutical preparation comprising the combination of agents of claim 9 in a biologically acceptable carrier.

11. The method of claim 1, wherein said cell line comprising a mutation in the CFTR is isolated from a cystic fibrosis patient.

12. The method of claim 12, wherein said cells comprising the Δ508 mutation in said CFTR.
